(19) United States
(12) Patent Application Publication
(10) Pub. No.: US 2012/0022087 A1
(43) Pub. Date: Jan. 26, 2012

(54) AMORPHOUS AMBRISENTAN

(75) Inventors: Kathrin Rimkus, Iserlohn (DE); Frank Muskulus, Laupheim (DE); Sandra Brueck, Ottenhofen (DE); Jana Paetz, Bonn (DE)

(73) Assignee: Ratiopharm GmbH, Ulm (DE)

(21) Appl. No.: 13/055,421

(22) PCT Filed: Aug. 7, 2009
(86) PCT No.: PCT/EP2009/005750

§ 371 Date: Sep. 15, 2011

(30) Foreign Application Priority Data
Aug. 11, 2008 (DE) 10 2008 037 325.7

(51) Int. Cl. A61K 31/505; A61P 9/12; B29C 37/00; C07D 239/34

(52) U.S. Cl. 514/274; 544/318; 264/140

(57) ABSTRACT

The invention relates to amorphous ambrisentan, preferably together with a surface stabiliser in the form of a stable intermediate. The invention further relates to methods of producing stable amorphous ambrisentan and pharmaceutical formulations containing stable amorphous ambrisentan.

The invention relates to amorphous ambrisentan, preferably together with a surface stabiliser in the form of a stable intermediate. The invention further relates to methods of preparing stable amorphous ambrisentan and pharmaceutical formulations containing stable amorphous ambrisentan.

Ambrisentan is an endothelin receptor antagonist and is approved for the treatment of pulmonary hypertension (high blood pressure in the lungs). As an antagonist, ambrisentan selectively displaces endothelin-1, the most powerful endogenous vasoconstrictor known, from its ET1A receptors and thus cancels out the effect of endothelin-1, so that the vessels dilate, in this way countering the increase in (pulmonary) blood pressure caused by the endothelin, leading in the process to a reduction in (pulmonary) blood pressure.

The IUPAC name for ambrisentan [1NN] is (2S)-2-(4,6-dimethylpyrimidin-2-yl)oxy-3-methoxy-3,3-di(phenyl)propanoic acid. The chemical structure of ambrisentan is shown in the (1) below:

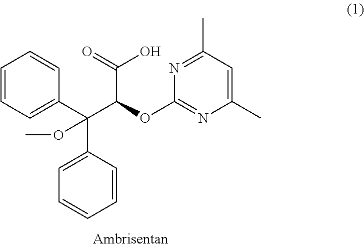

The synthesis of ambrisentan was described by Riechers et al, J. Med. Chem. 39 (11), 2123 (1996) and in WO 96/11914 and leads to a white, crystalline solid.

Ambrisentan is marketed under the trade name Volibris® as film-coated tablets. Volibris contains ambrisentan in crystalline form, with the tablets produced by means of direct compression (see EMEA “Assessment Report for Volibris”, 2008, Procedure No. EMEA/H/C/000839). In order to guarantee the necessary bioavailability, crystalline ambrisentan is preferably used in micronised form.

The micronisation of ambrisentan entails a number of disadvantages, however. First of all, the micronisation results in an active agent with undesirably poor flowability. In addition, the micronised active agent is more difficult to compress, and there is occasionally an uneven distribution of the active agent within the pharmaceutical formulation to be compressed. The considerable enlargement of the surface area during micronisation also causes the sensitivity of the active agent to oxidation to increase.

The problem of the present invention was therefore to overcome the above-mentioned disadvantages. The intention is to provide the active agent in a form which possesses good flowability and makes good compression possible. In addition, it is intended to enable an even distribution of the active agent. It is intended to avoid micronisation of the active agent.

The intention is also to provide the active agent in a form which possesses good solubility with good storage stability. In addition, it is intended to achieve a storage stability of 12 months at 40° C. and 75% atmospheric humidity. The impurities after storage under these conditions are intended to be <2% by weight, especially <1% by weight.

It was unexpectedly possible to solve the problems by converting crystalline ambrisentan into an amorphous state, especially into a stabilised amorphous state.

The subject matter of the invention is therefore amorphous ambrisentan in a stabilised form.

In particular, the subject matter of the invention is an intermediate containing amorphous ambrisentan and a surface stabiliser, preferably a polymer with a glass transition temperature (Tg) of higher than 25° C., wherein the weight ratio of ambrisentan to surface stabiliser is from 1:50 to 2:1. The intermediate is amorphous ambrisentan in stabilised form.

The subject matter of the invention also relates to various methods of preparing amorphous ambrisentan or stabilised amorphous ambrisentan in the form of the intermediate of the invention.

Finally, the subject matter of the invention comprises pharmaceutical formulations containing the ambrisentan stabilised in accordance with the invention in the form of the intermediate.

In the context of this invention, the term “ambrisentan” comprises (2S)-2-(4,6-dimethylpyrimidin-2-yl)oxy-3-methoxy-3,3-di(phenyl)propanoic acid in accordance with formula (1) above. In addition, the term “ambrisentan” comprises all the pharmaceutically acceptable salts and solvates thereof.

The term “amorphous” is used in the context of this invention to designate the state of solid substances in which the components (atoms, ions or molecules, i.e. in the case of amorphous ambrisentan the ambrisentan molecules) do not exhibit any periodic arrangement over a great range (=long-range order). In amorphous substances, the components are usually not arranged in a totally disordered fashion and completely randomly, but are rather distributed in such a way that a certain regularity and similarity to the crystalline state can be observed with regard to the distance from and orientation towards their closest neighbours (=short-range order). Amorphous substances consequently preferably possess a short-range order, but no long-range order.

In contrast to anisotropic crystals, solid amorphous substances are isotropic. Normally, they do not have a defined melting point, but instead pass over into the liquid state after slowly softening. They can be distinguished from crystalline substances experimentally by means of X-ray diffraction, which does not reveal clearly defined interferences for them, but rather, in most cases, only a few diffuse interferences with small diffraction angles.

In DSC analysis, crystalline ambrisentan exhibits the following characteristic peaks: 157° C. exothermic, 180° C. endothermic, 181° C. exothermic. The amorphous ambrisentan of the invention, on the other hand, usually exhibits a softening range from 40 to 70° C., preferably from 45 to 65° C. The melting point and softening range are determined in the context of this invention by means of dynamic differential scanning calorimetry (DSC).

The amorphous ambrisentan of the invention may consist of amorphous ambrisentan. Alternatively, it may also contain small amounts of crystalline ambrisentan components, provided that no defined melting point of crystalline ambrisentan can be detected in DSC. A mixture containing 60 to 99.999% by weight amorphous ambrisentan and 0.001 to 40% by weight crystalline ambrisentan is preferred, more preferably 90 to 99.99% by weight amorphous ambrisentan and 0.01 to 10% crystalline ambrisentan, particularly preferably 95 to 99.9% by weight amorphous ambrisentan and 0.1 to 5% crystalline ambrisentan.

In a preferred embodiment, the ambrisentan of the invention is present in stabilised form, namely in the form of an intermediate containing amorphous ambrisentan and a surface stabiliser. In particular, the intermediate of the invention consists substantially of amorphous ambrisentan and surface stabiliser. The expression “substantially” in this case indicates that small amounts of solvent etc. may also be present where applicable.

The surface stabiliser is generally a substance which inhibits the recrystallisation of amorphous to crystalline ambrisentan. The surface stabiliser is preferably a polymer. In addition, the surface stabiliser also includes substances which behave like polymers. Examples of these are fats and waxes. Furthermore, the surface stabiliser also includes solid, non-polymeric compounds which preferably contain polar side groups. Examples of these are sugar alcohols or disaccharides. Finally, the term “surface stabiliser” also encompasses surfactants, especially surfactants which are present in solid form at room temperature.

The polymer to be used for the preparation of the intermediate preferably has a glass transition temperature (Tg) of more than 25° C., more preferably 40° C. to 150° C., in particular from 50° C. to 100° C. By immobilisation, a polymer with a Tg selected accordingly is particularly advantageous in preventing the recrystallisation of the amorphous ambrisentan.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Beneath the Tg, a polymer is usually glassy and hard, whereas above the Tg, it changes into a rubber-like to viscous state. The glass transition temperature is determined in the context of this invention by means of dynamic differential scanning calorimetry (DSC).

For this purpose a Mettler Toledo DSC 1 apparatus can be used. The work is performed at a heating rate of 1-20° C./min, preferably 5-15° C./min, and at a cooling rate of 5-25° C./min, preferably 10-20° C./min.

In addition, the polymer to be used for the preparation of the intermediate preferably has a number-average molecular weight of 1,000 to 500,000 g/mol, more preferably from 2,000 to 50,000 g/mol. If the polymer used for the preparation of the intermediate is dissolved in water in an amount of 2% by weight, the resulting solution preferably has a viscosity of 1 to 20 mPa×s, more preferably either 1 to 5 mPa×s, and even more preferably from 2 to 4 mPa×s or (especially in the case of HPMC) from 12 to 18 mPa×s, measured at 25° C., and determined in accordance with Ph. Eur., 6th edition, chapter 2.2.10.

Hydrophilic polymers are preferably used for the preparation of the intermediate. This refers to polymers which possess hydrophilic groups. Examples of suitable hydrophilic groups are hydroxy, sulphonate, carboxylate and quaternary ammonium groups.

The intermediate of the invention may comprise the following polymers, for example: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC); polyvinyl pyrrolidone, polyvinyl alcohol, polymers of acrylic acid and their salts, vinyl pyrrolidone-vinyl acetate copolymers (such as Kollidon VA64, BASF), gelatine polyalkylene glycols, such as polypropylene glycol or preferably polyethylene glycol; gelatine and mixtures thereof.

It is likewise preferably possible to use sugar alcohols such as mannitol, sorbitol, xylitol as surface stabilisers. The waxes used are preferably cetyl palmitate, carnauba wax. The fats used are preferably glycerol fatty acid esters e.g. glycerol palmitate, behenate, laurate, stearate, PEG glycerol fatty acid ester.

The surface stabilisers preferably used are polyvinyl pyrrolidone, preferably with a number-average molecular weight of 10,000 to 60,000 g/mol, especially 12,000 to 40,000 g/mol, vinyl pyrrolidone and vinyl acetate copolymer, especially with a number-average molecular weight of 45,000 to 75,000 g/mol and/or polymers of acrylic acid and their salts, especially with a number-average molecular weight of 50,000 to 250,000 g/mol. In addition, HPMC is preferably used, especially with a number-average molecular weight of 20,000 to 90,000 g/mol and/or preferably a proportion of methyl groups of 10 to 35% and a proportion of hydroxy groups of 1 to 35%. Likewise, HPC is preferably used, especially with a number-average molecular weight of 50,000 to 100,000 g/mol. Also, polyethylene glycol with a number-average molecular weight of 2,000 to 40,000 g/mol, especially from 3,500 to 25,000 g/mol, is preferably used. Likewise, a polyethylene/polypropylene block copolymer is preferably used, wherein the polyethylene content is preferably 70 to 90% by weight. The polyethylene/polypropylene block copolymer preferably has a number-average molecular weight of 1,000 to 30,000 g/mol, more preferably from 3,000 to 15,000 g/mol. The number-average molecular weight is usually determined by means of gel permeation chromatography.

In a first particularly preferred embodiment, the surface stabiliser used is a copolymer of vinyl pyrrolidone and vinyl acetate, especially with a weight-average molecular weight of 45,000 to 75,000 g/mol. The copolymer can be characterised by the following structural formula (2):

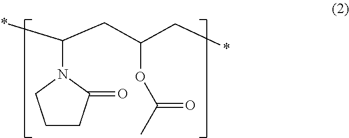

In a second particularly preferred embodiment, polymers of acrylic acid or salts thereof (also known as acrylic polymers) are used as surface stabilisers. In this case, it is preferably a polymer composed of structures according to the general formulae (4) and (3).

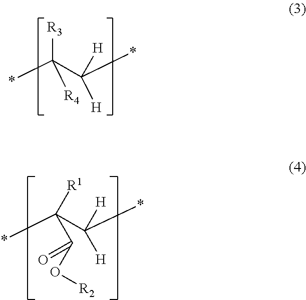

In formulae (4) and (3):

R₁ stands for a hydrogen atom or an alkyl radical, preferably a hydrogen atom or a methyl radical, especially a methyl radical;

R₂ stands for a hydrogen atom or an alkyl radical, preferably a hydrogen atom or a C₁ to C₄ alkyl radical, especially a methyl radical or an ethyl radical;

R₃ stands for a hydrogen atom or an alkyl radical, preferably a hydrogen atom or a methyl radical;

R₄ stands for an organic radical, preferably a carboxylic acid group or a derivative thereof, more preferably a group of the formula —COOH, —COOR₅,

R₅ stands for an alkyl radical or a substituted alkyl radical, preferably methyl, ethyl, propyl or butyl as an alkyl radical or —CH₂—CH₂—N(CH₃)₂ or —CH₂—CH₂—N(CH₃)₃_{⁺} halogen⁻ (especially Cl⁻) as a substituted alkyl radical.

The acrylic polymer contains structures in accordance with formulae (4) and (3), usually in molar ratios of 1:40 to 40:1. The ratio of structures according to formula (4) to structures according to formula (3) is preferably 2:1 to 1:1, especially 1:1. Where R₄ is —COO—CH₂—CH₂—N(CH₃)₃_{⁺}Cl⁻, the ratio of structures according to formula (4) to structures according to formula (3is) preferably 20:1 to 40:1.

If alternating polymerisation in the ratio 1:1 occurs, the result is preferably a polymer according to the formula (4+3)

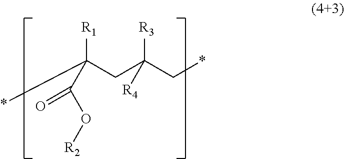

Polyacrylates according to the above formulae (4) and (3) are particularly preferred, where R₁ and R₃ is alkyl, especially methyl, R₂ is methyl or butyl, preferably methyl, and R₄ is —COO—CH₂—CH₂—N(CH₃)₂. In this case, the ratio of structures according to formula (2) to structures according to formula (3) is preferably 1:1. A corresponding polymer in particular has a number-average molecular weight of 50,000 to 250,000 g/mol, more preferably from 120,000 to 180,000 g/mol.

In a preferred embodiment, the intermediate of the invention contains amorphous ambrisentan and surface stabiliser, the weight ratio of ambrisentan to surface stabiliser being 1:50 to 2:1, more preferably 1:20 to 1:1, even more preferably 1:15 to 1:2, especially 1:12 to 1:5.

In a preferred embodiment, the intermediate of the invention is a “single-phase” intermediate. This means that the surface stabiliser and the amorphous ambrisentan are homogeneously distributed on the molecular level. In DSC analysis, the peaks characteristic of crystalline ambrisentan no longer occur at 157° C. exothermic, 180° C. endothermic and 181° C. exothermic.

It is preferable that the type and quantity of surface stabiliser should be selected such that the resulting intermediate has a glass transition temperature (Tg) of more than 20° C., preferably >40° C. The Tg of the intermediate should not be higher than 90° C.

It is preferable that the type and quantity of the polymer should be selected such that the resulting intermediate is storage-stable. “Storage-stable” means that in the intermediate of the invention, after storage for 3 years at 25° C. and 50% relative humidity, the proportion of crystalline ambrisentan—based on the total amount of ambrisentan—is no more than 60% by weight, preferably no more than 30% by weight, more preferably no more than 15% by weight, in particular no more than 5% by weight.

The intermediates of the invention are obtainable by a variety of preparation methods. Depending on the preparation method, the intermediates are obtained in different particle sizes. Normally, the intermediates of the invention are present in particulate form and have an average particle diameter (D₅₀) of 50 to 750 μm.

The expression “average particle diameter” refers in the context of this invention to the D₅₀ value of the volume-average particle diameter determined by means of laser diffractometry. In particular, a Malvern Instruments Mastersizer 2000 was used to determine the diameter (wet measurement with ultrasound 60 sec., 2,000 rpm, preferably shading 4 to 13%, preferably dispersion in liquid paraffin, the evaluation being performed according to the Fraunhofer model). The average particle diameter, which is also referred to as the D₅₀ value of the integral volume distribution, is defined in the context of this invention as the particle diameter at which 50% by volume of the particles have a smaller diameter than the diameter which corresponds to the D₅₀ value.

Similarly, 50% by volume of the particles than have a larger diameter than the D₅₀ value.

The subject matter of the invention is also a method of preparing the amorphous ambrisentan of the invention or the intermediate of the invention. In the following, six preferred embodiments of such a method will be explained.

In a first preferred embodiment, the invention relates to a freeze-drying process, i.e. a method of producing the amorphous ambrisentan of the invention, especially the intermediate of the invention, comprising the steps of
(a1) dissolving the crystalline ambrisentan and the surface stabiliser in a solvent or mixture of solvents, and
(b1) freeze-drying the solution from step (a1).

In step (a1), ambrisentan, preferably ambrisentan and the surface stabiliser described above, is dissolved, preferably completely dissolved, in a solvent or mixture of solvents.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and DMSO is used.

Suitable surface stabilisers in this embodiment are in particular modified celluloses, such as HPMC, and sugar alcohols, such as mannitol and sorbitol. Likewise, it is particularly preferable to use polyvinyl pyrrolidone, especially with the molecular weights specified above.

The solution from step (a1) is cooled to about 10 to 50° C. below freezing point (i.e. it is frozen). Then the solvent is removed by sublimation. This is preferably done when the conductivity of the solution is less than 2%. The sublimation temperature is preferably determined by the point of intersection of the product temperature and Rx-10° C. Sublimation is preferably effected at a pressure of less than 0.1 mbar.

After sublimation is complete, the lyophilised amorphous ambrisentan, preferably the lyophilised intermediate, is heated to room temperature.

The process conditions in this first embodiment are preferably selected such that the resulting intermediate particles have a volume-average particle diameter (D₅₀) of 5 to 250 μm, more preferably 20 to 150 μm, in particular 50 to 100 μm.

In a second preferred embodiment, the invention relates to a “pellet-layering process”, i.e. a method of producing the amorphous ambrisentan of the invention, especially the intermediate of the invention, comprising the steps of
(a2) dissolving the crystalline ambrisentan and the surface stabiliser in a solvent or mixture of solvents, and
(b2) spraying the solution from step (a2) onto a substrate core.

In step (a2), ambrisentan, preferably ambrisentan and the surface stabiliser described above, is dissolved, preferably completely dissolved, in a solvent or mixture of solvents.

Suitable solvents are. for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and DMSO is used.

Suitable surface stabilisers in this second embodiment are in particular modified celluloses, such as HPMC, sugar alcohols, such as mannitol and sorbitol, and polyethylene glycol, in particular polyethylene glycol with a molecular weight of 2,000 to 10,000 g/mol.

In step (b2), the solution from step (a2) is sprayed onto a substrate core. Suitable substrate cores are particles consisting of pharmaceutically acceptable excipients, especially “neutral pellets”. The preferable pellets used are those which are obtainable under the trade name Cellets® and which contain microcrystalline cellulose.

Step (b2) is preferably performed in a fluidised bed dryer, such as a Glatt GPCG 3 (Glatt GmbH, Germany).

The process conditions in this second embodiment are preferably selected such that the resulting intermediate particles have a volume-average particle diameter (D₅₀) of 50 to 750 μm, more preferably 100 to 500 μm.

In a third preferred embodiment, the invention relates to a method of producing the amorphous ambrisentan of the invention, especially the intermediate of the invention, comprising the steps of
(a3) dissolving the crystalline ambrisentan and the surface stabiliser in a solvent or mixture of solvents, and
(b3) spray-drying the solution from step (a3).

The third embodiment is particularly preferable.

In step (a3), ambrisentan, preferably ambrisentan and the surface stabiliser described above, is dissolved, preferably completely dissolved, in a solvent or mixture of solvents.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, a DMSO/water mixture is used.

Suitable surface stabilisers in this embodiment are in particular modified celluloses, such as HPMC, polyvinyl pyrrolidone and copolymers thereof, and sugar alcohols, such as mannitol and sorbitol. Acrylic polymers are likewise particularly preferable, especially the acrylic polymers described above under formulae (3) and (4).

In the subsequent step (b3), the solution from step (a3) is spray-dried. The spray-drying is usually carried out in a spray tower. As an example, a Büchi B-191 is suitable (Büchi Labortechnik GmbH, Germany). Preferably an inlet temperature of 100° C. to 150° C. is chosen. The amount of air is, for example, 500 to 700 litres/hour, and the aspirator preferably runs at 80 to 100%.

The process conditions in this third embodiment are preferably selected such that the resulting intermediate particles have a volume-average particle diameter (D₅₀) of 5 to 250 μm, more preferably 20 to 150 μm, in particular 50 to 100 μm.

In a fourth preferred embodiment, the invention relates to a melt extrusion process, i.e. a method of producing the intermediate of the invention, comprising the steps of
(a4) mixing crystalline ambrisentan and polymeric surface stabiliser, and
(b4) extruding the mixture.

In step (a4), crystalline ambrisentan is mixed with the surface stabiliser preferably in a mixer. In this embodiment of the method of the invention, a surface stabiliser in polymeric form is used.

Suitable polymeric surface stabilisers in this fourth embodiment are in particular polyvinyl pyrrolidone and copolymers thereof (especially a copolymer in accordance with the above formula (2)), and polyvinyl alcohols, methacrylates and HPMC.

Likewise, it is preferable to use polyethylene glycol, especially with the molecular weights specified above.

In step (b4), the mixture is extruded. For this purpose, conventional melt extruders can be used. By way of example, a Leistritz Micro 18 is used.

The cooled melt is comminuted by a rasp screen (e.g. Comill U5) and in this way reduced to a uniform particle size.

The extrusion temperature depends on the nature of the polymeric surface stabiliser. It is usually between 40 and 250° C., preferably between 80 and 160° C.

The cooled melt is preferably comminuted by a rasp screen and in this way reduced to a uniform particle size.

The process conditions in this fourth embodiment are preferably selected such that the resulting intermediate particles have a volume-average particle diameter (D₅₀) of up to 1,000 μm, more preferably a D₉₀ of 500 to 1,000 μm.

In a fifth preferred embodiment, the invention relates to a “hot-melt method”, i.e. a method of preparing the intermediate of the invention, comprising the steps of
(a5) incorporating crystalline ambrisentan into a surface stabiliser melt, and
(b5) applying the melt to a substrate pellet.

In step (a5), crystalline ambrisentan is dissolved, preferably completely dissolved, in a melt of the surface stabiliser. In this embodiment, waxes and fats are preferably used as surface stabilisers. One example of a preferably used surface stabiliser is Poloxamer®.

In step (b5), the melt from step (b2) is applied, preferably sprayed, onto a substrate core. Suitable substrate cores are particles consisting of pharmaceutically acceptable excipients, especially “neutral pellets”. The preferable pellets used are those which are obtainable under the trade name Cellets® and which contain a mixture of lactose and microcrystalline cellulose.

The process conditions in this fifth embodiment are preferably selected such that the resulting intermediate particles have a volume-average particle diameter (D₅₀) of 50 to 750 μm, more preferably 100 to 500 μm.

In a sixth preferred embodiment, the invention relates to a milling process, i.e. a method of preparing the intermediate of the invention, comprising the steps of
(a6) mixing crystalline ambrisentan and surface stabiliser, and
(b6) milling the mixture from step (a6), the milling conditions being selected such that there is a transition from crystalline to amorphous ambrisentan.

Crystalline ambrisentan and surface stabiliser are mixed in step (a6). The mixture is milled in step (b6). The mixing may take place before or even during the milling, i.e. steps (a6) and (b6) may be performed simultaneously.

The milling conditions are selected such that there is a transition from crystalline to amorphous ambrisentan.

The milling is generally performed in conventional milling apparatuses, preferably in a ball mill, such as a Retsch PM 100.

The milling time is usually 10 minutes to 10 hours, preferably 30 minutes to 8 hours, more preferably 2 hours to 6 hours.

Suitable surface stabilisers in this sixth embodiment are in particular modified celluloses, such as HPMC, sugar alcohols, such as mannitol and sorbitol, and polyethylene glycol, in particular polyethylene glycol with a molecular weight of 2,000 to 10,000 g/mol. Polyvinyl pyrrolidone is likewise preferably used.

The process conditions in this sixth embodiment are preferably selected such that the resulting intermediate particles have a volume-average particle diameter (D₅₀) of 5 to 250 μm, more preferably 10 to 150 μm, especially 20 to 80 μm or 20 to 150 μm, more preferably 50 to 100 μm.

The amorphous ambrisentan of the invention and the intermediate of the invention (i.e. the stabilised amorphous ambrisentan of the invention) are usually employed to prepare a pharmaceutical formulation.

The subject matter of the invention is therefore a pharmaceutical formulation containing amorphous ambrisentan of the invention or the intermediate of the invention and pharmaceutical excipients.

These are the excipients with which the person skilled in the art is familiar, such as those which are described in the European Pharmacopoeia.

Examples of excipients used are disintegrants, anti-stick agents, pseudo-emulsifiers, fillers, additives to improve the powder flowability, glidants, wetting agents, gelling agents and/or lubricants.

The ratio of active agent to excipients is preferably selected such that the resulting formulations contain

1 to 50% by weight, more preferably 2 to 30% by weight, in particular 5 to 20% by weight amorphous ambrisentan and

50 to 99% by weight, more preferably 70 to 98% by weight, in particular 80 to 95° AI by weight pharmaceutically acceptable excipients.

In these ratios specified, the amount of surface stabiliser optionally used to prepare the intermediate of the invention is counted as an excipient. This means that the amount of active agent refers to the amount of amorphous ambrisentan contained in the intermediate.

It has become apparent that a large amount of disintegrants is particularly preferable in solving the problems described above.

In a preferred embodiment, the pharmaceutical formulation of the invention therefore contains

(i) 1 to 50% by weight, more preferably 2 to 30% by weight, in particular 5 to 20% by weight amorphous ambrisentan and

(ii) 5 to 30% by weight, more preferably 2 to 25% by weight, in particular 3 to 15% by weight or 5 to 30% by weight, more preferably 10 to 25% by weight, in particular 12 to 22% by weight disintegrants, based on the total weight of the formulation.

In addition, the pharmaceutical formulation preferably contains one or more of the above-mentioned excipients.

“Disintegrants” is the term generally used for substances which accelerate the disintegration of a dosage form, especially a tablet, after it is placed in water. Suitable disintegrants are, for example, organic disintegrants such as carrageenan, croscarmellose, sodium carboxymethyl starch and crospovidone. Alkaline disintegrants are preferably used. The term “alkaline disintegrants” means disintegrants which, when dissolved in water, produce a pH level of more than 7.0.

More preferably, inorganic alkaline disintegrants are used, especially salts of alkali metals and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

Sodium hydrogen carbonate is particularly preferably used as a disintegrant, especially in the above-mentioned amounts.

In a further preferred embodiment, the pharmaceutical formulation additionally contains

(iii) anti-stick agents, preferably in an amount of 0.1 to 5% by weight, more preferably 0.5 to 3% by weight, based on the total weight the formulation.

“Anti-stick agents” are usually understood to mean substances which reduce agglomeration in the core bed. Examples are talcum, silica gel, polyethylene glycol (preferably with 2,000 to 10,000 g/mol weight-average molecular weight) and/or glycerol monostearate.

Examples of preferred anti-stick agents are talcum and polyethylene glycol 4,000, agar and/or carrageenan.

In a further preferred embodiment, the pharmaceutical formulation additionally contains an

(iv) emulsifier and/or pseudo-emulsifier, preferably in an amount of 0.1 to 5% by weight, more preferably 0.5 to 3% by weight, based on the total weight of the formulation.

Pseudo-emulsifiers are usually (preferably polymeric) substances which, when added to a solution, increase the viscosity of that solution. Preferably, the addition of 5% by weight of pseudo-emulsifier to distilled water at 20° C. leads to an increase in the viscosity of at least 1%, preferably at least 2%, in particular at least 5%.

Plant gums are preferably used as pseudo-emulsifiers. Plant gums are polysaccharides of natural origin which cause the above-mentioned viscosity increase.

Examples of suitable pseudo-emulsifiers are agar, alginic acid, alginate, chicle, dammar, mallow extracts, gellan (E 418), guar gum (E 412), gum arabic (E 414), gum from psyllium seed husks, gum from spruce resin, locust bean gum (E 410), karaya (E 416), glucomannan (E 425), obtained from the konjac root, tara gum (E 417), gum traganth (E 413), xanthan gum (E 415), preferably prepared by bacterial fermentation, and/or lecithin.

Gum arabic, agar and/or lecithin are preferably used.

Possible emulsifiers are anionic emulsifiers, e.g. □soaps, preferably alkali salts of higher fatty acids □salts of bile acid (alkali salts); cation-active emulsifiers, e.g. □benzalconium chloride, □cetyl pyridinium chloride, □cetrimide; non-ionic emulsifiers, e.g. □sorbitan derivatives, especially sorbitan monolaurate, polyoxythylene-(20)-sorbitan-monolaurate, □polyethylene glycol derivatives/polyoxyethylene derivative, especially polyoxyethylene-(20)-sorbitan monostearate, polyoxythylene stearate or polyoxyethylene stearyl ether. In addition, partial fatty acid esters of polyhydric alcohols can be used, such as glycerol monostearate, fatty acid ester of sucrose, □fatty acid ester of polyglycol or □casein. Similarly, mixtures of the above-mentioned substances are possible.

In addition to components (i) to (iv), the formulation of the invention may also contain further, above-mentioned pharmaceutical excipients. These will be explained in more detail below.

The formulation of the invention preferably contains fillers. “Fillers” generally means substances which serve to form the body of the tablet in the case of tablets with small amounts of active agent (e.g. less than 70% by weight). This means that fillers “dilute” the active agents in order to produce an adequate tablet-compression mixture. The normal purpose of fillers, therefore, is to obtain a suitable tablet size.

Examples of preferred fillers are lactose, lactose derivatives, starch, starch derivatives, treated starch, talcum, calcium phosphate, hydrogen phosphate sucrose, calcium carbonate, magnesium carbonate, magnesium oxide, maltodextrin, calcium sulphate, dextrates, dextrin, dextrose, hydrogenated vegetable oil, kaolin, sodium chloride, and/or potassium chloride. ProsoIv® (Rettenmaier & Söhne, Germany) can also be used.

Fillers are generally used in an amount of 1 to 80% by weight, more preferably 15 to 70% by weight, particularly preferably 30 to 60% by weight, based on the total weight of the formulation.

One example of an additive to improve the powder flowability is disperse silicon dioxide, e.g. known under the trade name Aerosil®. Preferably, silicon dioxide is used with a specific surface area of 50 to 400 m²/g, determined by gas adsorption in accordance with Ph. Eur., 6th edition 2.9.26.

Additives to improve the powder flowability are generally used in an amount of 0.1 to 3% by weight, based on the total weight of the formulation.

In addition, lubricants may be used. Lubricants are generally used in order to reduce sliding friction. In particular the intention is to reduce the sliding friction found during tablet pressing between the punch moving up and down in the die and the die wall, on the one hand, and between the edge of the tablet and the die wall, on the other hand. Suitable lubricants are, for example, stearic acid, adipic acid, sodium stearyl fumarate and/or magnesium stearate.

Lubricants are generally used in an amount of 0.1 to 3% by weight, based on the total weight of the formulation.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient. For example, PEG 4000—if used as a surface stabiliser—is not additionally used as an anti-stick agent (even though PEG 4000 also exhibits a release effect). Similarly, microcrystalline cellulose—if used as a surface stabiliser—is not also used as a disintegrant, for example (even though microcrystalline cellulose also exhibits a certain disintegrating effect).

The pharmaceutical formulation of the invention is preferably pressed into tablets. In the state of the art, direct pressing of an ambrisentan formulation is proposed (cf. EMEA “Assessment Report for Volibris”, 2008, Procedure No. EMEA/H/C/000839). It has, however, become apparent that the properties of the resulting tablets can be improved if the pharmaceutical formulation of the invention is subjected to dry granulation before being pressed into a tablet.

The subject matter of the present invention is therefore a method comprising the steps of
(I) preparing the amorphous ambrisentan of the invention or the intermediate of the invention and one or more pharmaceutical excipients (especially those described above);
(II) compacting it into flakes; and
(III) granulating or comminuting the flakes.

In step (I), ambrisentan and excipients are preferably mixed. The mixing can be performed in conventional mixers. Alternatively, it is possible that the amorphous ambrisentan is initially only mixed with part of the excipients (e.g. 50 to 95%) before compacting (II), and that the remaining part of the excipients is added after the granulation step (III). In the case of multiple compacting, the excipients should preferably be mixed in before the first compacting step, between multiple compacting steps or after the last granulation step.

In step (II) of the method of the invention, the mixture from step (I) is compacted into flakes. It is preferable here that it should be dry compacting, i.e. the compacting is preferably performed in the absence of solvents, especially in the absence of organic solvents.

The compacting conditions in step (II) are preferably selected such that the flakes have a density of 1.03 to 1.3 g/cm³, especially 1.05 to 1.2 g/cm³.

The term “density” here preferably relates to the “pure density” (i.e. not to the bulk density or tamped density). The pure density can be determined with a gas pycnometer. The gas pycnometer is preferably a helium pycnometer; in particular, the AccuPyc 1340 helium pycnometer from the manufacturer Micromeritics, Germany, is used.

The compacting is preferably carried out in a roll granulator.

The rolling force is preferably 2 to 50 kN/cm, more preferably 4 to 30 kN/cm, especially 10 to 25 kN/cm.

The gap width of the roll granulator is, for example, 0.8 to 5 mm, preferably 1 to 4 mm, more preferably 1.5 to 3 mm, especially 1.8 to 2.8 mm.

The compacting apparatus used preferably has a cooling means. In particular, the cooling is such that the temperature of the compacted material does not exceed 50° C., especially 40° C.

In step (iii) of the method the flakes are granulated. The granulation can be performed with methods known in the state of the art.

In a preferred embodiment, the granulation conditions are selected such that the resulting particles (granules) have a volume-average particle size (d(₅₀) value) of 50 to 600 μm, more preferably 100 to 500 μm, even more preferably 150 to 400 μm, especially 200 to 350 μm.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

In a preferred embodiment, the method is adapted such that multiple compacting occurs, with the granules resulting from step (III) being returned one or more times to the compacting (II). The granules from step (III) are preferably returned 1 to 5 times, especially 2 to 3 times.

The granules resulting from step (III) can be further processed into pharmaceutical dosage forms. For this purpose, the granules are filled into sachets or capsules, for example. The granules resulting from step (III) are preferably pressed into tablets (IV).

In step (IV) of the method, the granules obtained in step (III) are pressed into tablets, i.e. the step involves compression into tablets. The compression can be performed with tableting machines known in the state of the art.

In step (IV) of the method, pharmaceutical excipients may optionally be added to the granules from step (III).

The amounts of excipients added in step (IV) usually depend on the type of tablet to be produced and the amount of excipients which were already added in steps (I) or (II). The tableting conditions are preferably selected such that the resulting tablets have a ratio of tablet height to weight of 0.005 to 0.3 mm/mg, particularly preferably 0.05 to 0.2 mm/mg.

In addition, the resulting tablets preferably have a hardness of 35 or 50 to 200 N, particularly preferably 60 or 80 to 150 N. The hardness is determined in accordance with Ph. Eur. 6.0, section 2.9.8.

In addition, the resulting tablets preferably have a friability of less than 10%, particularly preferably less than 5%, especially less than 3%. The friability is determined in accordance with Ph. Eur. 6.0, section 2.9.7.

Finally, the tablets of the invention usually have a “content uniformity” of 85 to 115% preferably 90 to 110%, especially 95 to 105% of the average content. The “content uniformity” is determined in accordance with Ph. Eur.6.0, section 2.9.6.

The release profile of the tablets of the invention according to the USP method after 10 minutes usually indicates a content release of at least 30%, preferably at least 50%, especially at least 70%.

The above details regarding hardness, friability, content uniformity and release profile preferably relate here to the non-film-coated tablet.

The tablets produced by the method of the invention may be tablets which can be swallowed unchewed (non-film-coated or preferably film-coated). They may likewise be chewable tablets or dispersible tablets. “Dispersible tablet” here means a tablet to be used for producing an aqueous suspension for swallowing.

In the case of tablets which are swallowed unchewed, it is preferable that they be coated with a film layer. For this purpose, the methods of film-coating tablets which are standard in the state of the art may be employed. The above-mentioned ratios of active agent to excipient, however, relate to the uncoated tablet.

For film-coating, macromolecular substances are preferably used, such as modified celluloses, polymethacrylates, polyvinyl pyrrolidone, polyvinyl acetate phthalate, zein and/or shellack.

HPMC is preferably used, especially HPMC with a number-average molecular weight of 10,000 to 150,000 g/mol and/or an average degree of substitution of —OCH₃ groups of 1.2 to 2.0.

The thickness of the coating is preferably 10 to 100 μm.

The invention will now be explained with reference to the following examples.

### EXAMPLES

### Example 1

### Preparation of the Intermediate by Milling

5 g crystalline ambrisentan were co-milled with 25 g HPMC in a PM 100 ball mill (ex Retsch) for 2-3 hours at a speed of 350 rpm.

### Example 2

### Preparation of the Intermediate by Lyophilisation

5 g crystalline ambrisentan were dissolved with 10 g mannitol in DMSO/water and frozen at −50° C. until no electric conductivity any more was measurable. After that, the solvent was sublimed at a temperature of 10° C. below the eutectic temperature of the mixture under a 1 mbar vacuum. When no change in the pressure could be detected any more, the mixture was slowly raised to room temperature.

### Example 3

### Preparation of the Intermediate by Melt Extrusion

5 kg crystalline ambrisentan were pre-mixed with 50 kg copolymer polyvinyl pyrrolidone and polyvinyl acetate (Povidon® VA 64, BASF). This mixture was extruded on a twin-screw extruder with a temperature cascade rising to 150° C. (Leistritz Micro 18). The cooled strands were then Comill-screened.

### Example 4

### Preparation of the Intermediate by Pellet-Layering

100 g crystalline ambrisentan were dissolved in a water/DMSO solution and sprayed as a solution together with 500 g PEG 4000 onto inert Cellets (ethyl cellulose pellets).

This work was done in the “Heinen Minibatch”. Inlet air temperature 60-80° C., product temperature 30-40° C., spray pressure 1-2.5 bar, nozzle 1-2 mm.

### Example 5

### Preparation of the Intermediate by “Hot-Melt Coating”

50 g crystalline ambrisentan were dissolved in 700 g melted Gelucire® (fatty acid glycerol PEG ester) at 60° C. This melt was applied to “sugar spheres” using the hot-melt method:

For this purpose, the work was done with a “Müttlin spheric coater Unilab-05/-5-TJ”: inlet air temperature 250° C., microclimate 100° C., spray pressure 0.4 bar.

### Example 6

### Preparation of the Intermediate by Spray-Drying

10 g crystalline ambrisentan were dissolved in water/DMSO with 20 g Povidon 25 and 10 g lactose. The solution was spray-dried in the “Büchi”. For this purpose, the following parameters were set: aspirator 95%, air flow 700 m³/h, inlet air 130° C.

### Example 7

### Production of Tablets

In order to produce tablets, the following formulation was used:

| | |
|---|---|
| 1. intermediate according to example 6 | 30 g |
| 2. talcum | 1 g |
| 3. siliconised microcrystalline cellulose) | 90 g |
| 4. sodium hydrogen carbonate | 25 g |
| 5. silicon dioxide | 0.5 g |
| 6. Na-stearyl fumarate | 1 g |

Ingredients 1 and 2 were pre-mixed for 5 min in a free-fall mixer (Turbula TB 10). This mixture was compacted with 70% of ingredients 3-5 using a roll compactor and screened to 1.25 mm. The compacted material was mixed with the remaining substances and pressed into tablets.

### Example 8

### Preparation of the Intermediate by Milling

5.12 g ambrisentan and 10.00 g polyvinyl pyrrolidone (Mw 25 kDa) were mixed in the Turbula® T10B and milled for two hours (at 350 rpm, Retsch mill, PM100, 4 balls).

### Example 9

### Preparation of the Intermediate by Lyophilisation

5.12 g ambrisentan and 10.00 g polyvinyl pyrrolidone (Mw 25 kDa) and 800.00 g phosphate buffer (pH 7.4) were weighed together and the solution was stirred for 30 min. in a magnetic stirrer. Lyophilisation was carried out with a Christ Epsilon 2-4.

### Example 10

### Preparation of the Intermediate by Melt Extrusion

0.26 g ambrisentan and 0.50 g PEG 20000 were processed analogously to Example 3.

### Example 11

### Preparation of the Intermediate by Melt Extrusion

0.26 g ambrisentan and 0.50 g PEG 4000S were processed analogously to Example 3.

### Example 12

### Preparation of the Intermediate by Melt Extrusion

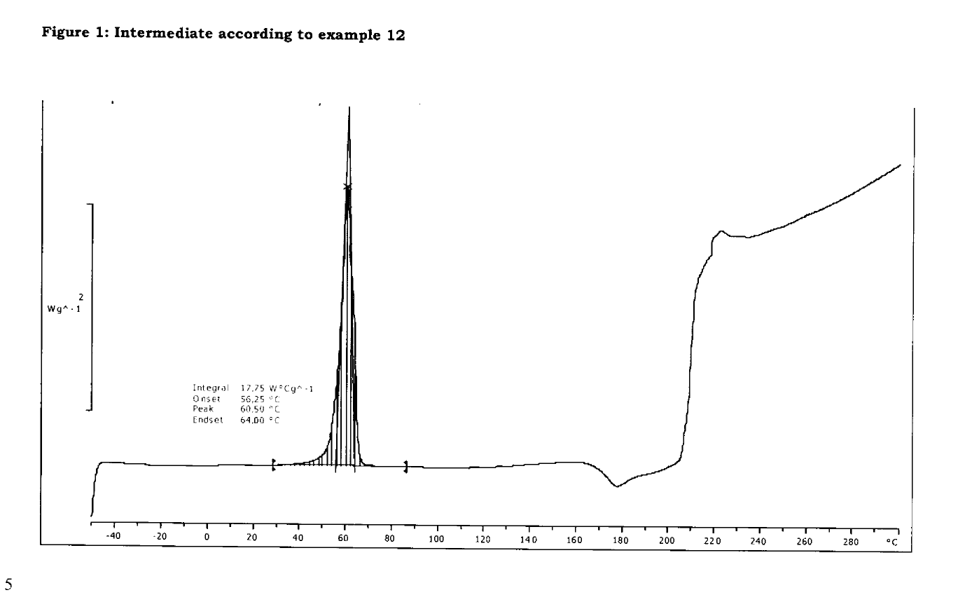

0.26 g ambrisentan and 0.50 g Pluronic F68 (Pluronic=PEG-PPO block copolymer) were processed analogously to Example 3. A DSC of the resulting amorphous ambrisentan intermediate is shown in FIG. 1.

### Example 13

### Melt (in the DSC Crucible)

Various binary mixtures of ambrisentan and polymer were prepared in a quantity ratio of 1:5. The mixtures were heated at a heating rate of 10° C./minute, tempered for 3-5 minutes and then cooled quickly to −50° C.

Mixture with Eudragit EPO: heated to 160° C., cooling rate 50° C./min

Kollidon® 25: heated to 160° C., cooling rate 50° C./min

Kollidon® VA 64: heated to 145° C., cooling rate 30° C./min

Klucel® (=HPC) heated to 160° C., cooling rate 50° C./min

### Example 14

### Preparation of the Intermediate by Spray-Drying

0.64 g ambrisentan and 6.25 g Eudragit® EPO were dissolved together in 250 g HCl buffer (pH 1.2) and then spray-dried.

### Example 15

### Production of Tablets

3.65 g intermediate according to example 14

4.66 g calcium hydrogen phosphate

0.18 g sodium carboxymethyl starch

0.66 g sodium hydrogen carbonate

0.09 g magnesium stearate

0.09 g talcum

0.41 g sodium stearyl fumarate

0.09 g Aerosil® (SiO₂)

The intermediate according to Example 14, calcium hydrogen phosphate, sodium carboxymethyl starch and sodium hydrogen carbonate were mixed together for 20 minutes and screened. In addition, magnesium stearate was added and mixed for 3 minutes. After that, talcum, sodium stearyl fumarate and Aerosil® were added and mixed for a further 3 minutes. The mixture was used to press tablets of 149 mg (containing 5 mg ambrisentan).

1. An intermediate containing amorphous ambrisentan and a surface stabiliser, the weight ratio of ambrisentan to surface stabiliser being 1:50 to 2:1.

2. An intermediate containing amorphous ambrisentan and a surface stabiliser characterised in that the surface stabiliser is a polymer, preferably a polymer with a glass transition temperature (Tg) of more than 25° C.

3. The intermediate as claimed in claim 1, characterised in that it is a single-phase intermediate.

4. The intermediate as claimed in claim 1 characterised in that the glass transition temperature (Tg) of the intermediate is more than 20° C.

5. A method of preparing an intermediate as claimed in claim 1 comprising the steps of
(a1) dissolving crystalline ambrisentan and surface stabiliser in a solvent or mixture of solvents, and
(b1) freeze-drying the solution from step (a1).

6. A method of preparing an intermediate as claimed in claim 1 comprising the steps of
(a2) dissolving crystalline ambrisentan and the surface stabiliser in a solvent or mixture of solvents, and
(b2) spraying the solution from step (a2) onto a substrate core.

7. A method of preparing an intermediate as claimed in claim 1 comprising the steps of
(a3) dissolving crystalline ambrisentan and the surface stabiliser in a solvent or mixture of solvents, and
(b3) spray-drying the solution from step (a3).

8. A method of preparing an intermediate as claimed in claim 1 comprising the steps of
(a4) mixing crystalline ambrisentan and surface stabiliser,
and
(b4) extruding the mixture.

9. A method of preparing an intermediate as claimed in claim 1 comprising the steps of
(a5) incorporating crystalline ambrisentan into a melt of the surface stabiliser, and
(b5) applying the melt to a substrate pellet.

10. A method of preparing an intermediate as claimed in claim 1 comprising the steps of
(a6) mixing crystalline ambrisentan and surface stabiliser, and
and
(b6) milling the mixture from step (a6), the milling conditions being selected such that there is a transition from crystalline to amorphous ambrisentan.

11. An intermediate obtainable by a method as claimed in claim 5.

12. A pharmaceutical formulation containing amorphous ambrisentan in the form of an intermediate as claimed in claim 1.

13. The pharmaceutical formulation as claimed in claim 12, containing
(i) 1 to 50% by weight amorphous ambrisentan and
(ii) 3 to 25% by weight disintegrants, based on the total weight of the dosage form.

14. The pharmaceutical formulation as claimed in claim 13, characterised in that it is an alkaline disintegrant, especially sodium hydrogen carbonate.

15. The pharmaceutical formulation as claimed in claim 12, containing
(iii) 0.1 to 5% by weight anti-stick agent.

16. The pharmaceutical formulation as claimed in claim 12, containing
(iv) 0.1 to 5% by weight emulsifier and/or pseudo-emulsifier, based on the total weight of the dosage form.

17. The pharmaceutical formulation as claimed in claim 12, obtainable by dry granulation.

18. A method of preparing a pharmaceutical formulation comprising the steps of
(I) providing the amorphous ambrisentan as claimed in claim 1 and one or more pharmaceutical excipients;
(II) compacting it into flakes; and
(III) granulating the flakes.

19. Tablets obtainable by compression of a pharmaceutical formulation as claimed in claim 12.

20. An intermediate obtainable by a method as claimed in claim 6.

21. An intermediate obtainable by a method as claimed in claim 7.

22. An intermediate obtainable by a method as claimed in claim 8.

23. An intermediate obtainable by a method as claimed in claim 9.

24. An intermediate obtainable by a method as claimed in claim 10.
